(19) United States
(12) Patent Application Publication
(10) Pub. No.: US 2020/0383585 A1
(43) Pub. Date: Dec. 10, 2020

(54) NON-INVASIVE MEASUREMENT OF MYOCARDIAL POWER

(71) Applicant: CHARITÉ - UNIVERSITÄTSMEDIZIN BERLIN, BERLIN (DE)

(72) Inventors: MARCUS KELM, BERLIN (DE); LEONID GOUBERGRITS, BERLIN (DE); JOAO FILIPE FERNANDES, LISBOA (PT); TITUS KÜHNE, BERLIN (DE)

(21) Appl. No.: 16/971,052

(22) PCT Filed: Feb. 22, 2019
(86) PCT No.: PCT/EP2019/054480

§ 371 Date: Aug. 19, 2020

(30) Foreign Application Priority Data
Feb. 23, 2018 (EP) 18158410.3

(51) Int. Cl. A61B 5/02; A61B 5/022; A61B 6/03; A61B 6/00; A61B 8/08; A61B 8/14; A61B 5/055; A61B 5/00; A61B 8/06; A61B 8/00

(52) U.S. Cl. CPC A61B 5/02028; A61B 5/02225; A61B 6/032; A61B 6/503; A61B 8/0883; A61B 8/14; A61B 5/024; A61B 5/055; A61B 5/0035; A61B 5/0044; A61B 8/065; A61B 8/0858; A61B 8/463; A61B 8/488

(57) ABSTRACT

A method for determining an internal myocardial power of a heart, the method comprising non-invasively obtaining one or more first images that are indicative of a structure of the heart, non-invasively obtaining one or more second images that are indicative of a flow property of the heart, segmenting, based on the one or more first images, the blood pool and the myocardium of the heart, determining, based on the segmented 5 blood pool, the myocardium and the one or more second images, one or more mechanical parameters of the heart, and determining the internal myocardial power based on the one or more mechanical parameters.

### TECHNICAL FIELD

The present invention relates to methods and devices for measuring myocardial power. The present invention also relates to a computer-readable storage medium storing program code, the program code comprising instructions for carrying out such a method.

### BACKGROUND

In heart disease pressure-volume overload conditions trigger cardiac remodeling which causes concentric or eccentric hypertrophy. At longer-term, there can be a transition from adaptive hypertrophy to heart failure. In addition, arrhythmias with risk for sudden cardiac death can evolve. However, there is large variability how patients deal with overload. Furthermore, neither the onset of clinical symptoms nor the degree of hypertrophy (chamber size, wall thickness) is directly related to the degree of external load on the heart.

Therefore, energetic approaches for evaluating the myocardial performance are becoming increasingly of interest. There is growing evidence that in disease the myocardial efficiency is reduced. This means that the myocardium will need more energy to pump a given amount of blood against a given resistance. Accordingly, new pharmacological or interventional therapeutic concepts aiming to enhance the myocardial efficiency and/or reduce the energy demand could be proposed.

Myocardial energy demand and efficiency are affected, amongst others, by adaptive changes in myocardial mass, LV chamber size and interstitial fibrosis in order to maintain myocardial wall stress according the Law of Laplace.

Methods for measuring myocardial efficiency did not become part of clinical routine yet due to their invasiveness. Current clinical routine is focusing on the assessment of the external myocardial power without an assessment of the internal myocardial power and respectively myocardial efficiency. More recently, combined Magnetic Resonance Imaging (MRI) and Positron Emission Tomography (PET) have been used to describe the ratio of the myocardial external power and internal energy consumption. While PET itself is non-invasive, it can only be applied for selected clinical indications and involves exposure to ionizing radiation. Thus the method is not available for the clinical routine.

### SUMMARY OF THE INVENTION

The objective of the present invention is to provide a method and a device for measuring myocardial power, wherein the method and the device overcome one or more of the above-mentioned problems of the prior art.

A first aspect of the present invention provides method for determining an internal myocardial power of a heart, the method comprising:
non-invasively obtaining one or more first images that are indicative of a structure of the heart,
non-invasively obtaining one or more second images that are indicative of a flow property of the heart,
segmenting, based on the first images, the blood pool and the myocardium of the heart,
determining, based on the segmented blood pool, the myocardium and the second images, one or more mechanical parameters of the heart, and
determining the internal myocardial power based on the one or more mechanical parameters.

The method of the first aspect allows determining an internal myocardial power of the heart without exposing the patient to radiation and without requiring invasive procedures such as placing a catheter. Experiments have shown that the method of the first aspect nonetheless provides clinically reliable accuracy.

Herein, internal myocardial power may refer to the power that the heart has to perform, in contrast to the external myocardial power that indicates how much power the heart is producing externally. The ratio between internal and external power is referred to as myocardial power efficiency.

Segmenting the myocardium of the heart may refer to segmenting the myocardium volume of the heart.

Thus, diagnostic measures of myocardial function based on non-invasive imaging data, which could be acquired in clinical routine.

Non-invasively obtaining images may include obtaining images over a network that have previously been acquired using a non-invasive imaging method (e.g. MRI or CT). Due to the required injection of a tracer, PET shall herein be considered as an invasive imaging method.

The one or more mechanical parameters of the heart may be any kind of parameters of the heart or a surrounding of the heart that relate to any kind of mechanical properties, e.g. a size, a volume, a duration, a duration of a periodic movement, a contraction time of a vehicle, an inflow closing time, or any other parameters relating to measured times or velocities of elements of the heart.

The first images may comprise one or more images of the heart at a first point in time and one or more images of the heart at a second point in time. Segmenting the first images may comprise performing a first segmentation of the images of the first point in time and performing a second segmentation of the images of the second point in time. These first, second (and potentially many further segmentations) may in the following just be referred to as “the segmentation”.

In embodiments, the internal myocardial power may also refer to a circulatory power of the heart. In embodiments, internal myocardial power may refer to the left ventricular (LV) internal myocardial power, as the mechanical power of the myocardium required to perform contraction generating LV peak systole pressure.

In a first implementation of the method of the first aspect, the method further comprises during the non-invasive acquisition of the first and second images, acquiring a heart rate and/or a systemic blood pressure. This has the advantage that the myocardial power can be determined with higher accuracy.

In a further implementation,
the first images are acquired using MRI, CT and/or echocardiography, and/or
the second images are acquired using 4D flow MRI, 2D flow MRI and/or Doppler echocardiography.

These imaging modalities have proven particularly useful for implementing the method of the first aspect.

In a further implementation, the one or more mechanical parameters include a contraction time of the heart, wherein the contraction time is determined based on a sum of an inflow valve closing time, an ejection time, and an isovolumetric contraction time.

Preferably, the inflow valve closing time is determined as a time when a first and a second pressure curve intersect, wherein preferably the first pressure curve is modeled as a hyperbola and the second curve is modeled as a parabola, the ejection time is determined based on an outflow curve that is determined based on the second images, and/or the isovolumetric contraction time is determined based on a combined inflow and outflow curve. This way of determining the inflow valve closing time has the advantage that the determination can be performed non-invasively.

In a further implementation, the one or more mechanical parameters include a ventricular radius, a length, a volume, a myocardial volume, and/or a myocardial wall thickness of the heart.

In a further implementation, the internal myocardial power that is determined based on a volume of the myocardial wall volume V_{wall}, a wall stress σ_{wall}, and a contraction time of the ventricle t_{cs}, preferably based on the equation

$$IMP = \frac{{V}_{wall} * {σ}_{wall}}{{t}_{C S}} .$$

This equation is easy to calculate, yet provides an accurate estimate of the internal myocardial power.

In a further implementation, the method further comprises determining a circulatory power based on a mean arterial pressure and an effective cardiac output, preferably based on a product of mean arterial pressure and effective cardiac output.

In a further implementation, the one or more mechanical parameters include a myocardial volume, a ventricular wall stress, a peak systolic pressure, a forward flow value and/or a backward flow volume.

In a further implementation, the method further comprises
non-invasively obtaining a systemic blood pressure based on a non-invasive blood pressure measurement, in particular a sphygmomanometer or oscillometric approach, and
determining, based on a flow property of the heart and the systemic blood pressure , one or more contraction time parameters of the heart.

In this implementation, the method may further comprise determining the internal myocardial power based on the one or more mechanical parameters, a myocardium volume of the heart and the one or more contraction time parameters of the heart. The myocardium volume may be determined based on the segmentation of the myocardium.

This implementation has the advantage that it allows a non-invasive assessment of the contraction time. Contraction time is a key parameter determining the heart power required to perform a certain work. Contraction time could also be directed measured during a heart catheterization procedure of a patient as it was shown earlier. Catheterization procedure is, however, invasive and associated with a radiation. Thus the catheterization method is not available for the majority of patients and cannot be used routinely in clinical practice.

A second aspect of the present invention provides a method for determining a remodeling of the heart of a patient, wherein the method comprises:
reading a plurality of images of the heart,
determining an internal myocardial power of the heart based on the plurality of images, and
determining a remodeling of the heart when the internal myocardial power is higher than a predetermined threshold, in particular a threshold between 5 and 8 W/m², preferably a threshold between 6 and 7 W/m².

Experiments have shown that the internal myocardial power (IMP) is a very accurate predictor of a remodeling of the heart of the patient. Herein, remodeling of the heart may refer to changes in the size, shape, structure, and function of the heart that are due to injury to the heart muscle (pathological remodeling).

Preferably, the internal myocardial power is determined using a method of the first aspect or one of the implementations of the first aspect. This has the advantage that the IMP can be determined non-invasively and with high accuracy.

Preferably, the plurality of images of the heart is read from a memory. Preferably, the method of the second aspect is performed in silico, i.e., not on a human or animal. In addition to reading the plurality of images of the heart from a memory, also additional parameters of the patient, e.g. a blood pressure measurement, may be read from a memory.

A third aspect of the present invention provides device for determining an internal myocardial power of a heart based on non-invasive imaging, the device comprising:
an image obtaining unit configured to obtain one or more first images of the heart that are indicative of a structure of the heart and one or more second images that are indicative of a flow property of the heart,
a segmentation unit configured to segment, based on the one or more first images, the blood pool and the myocardium of the heart,
a parameter determining unit configured to determine, based on the segmented blood pool and the myocardium and the one or more second images, one or more mechanical parameters, and
a calculation unit configured to determine the internal myocardial power based on the mechanical parameters.

In a first implementation of the third aspect, the image obtaining unit of the device comprises a network interface configured to obtain the first images and/or the second images from a remote device in the network, in particular from an MRI, CT or echocardiography machine.

In a second implementation of the device of the third aspect, the device further comprises a user interface for a visual verification of the segmentation by a physician. This has the advantage that potential mistakes by an automatic segmentation can be corrected.

A fourth aspect of the present invention provides a use of the device of the third aspect for determining a remodeling of the heart of a patient, comprising steps of
reading a plurality of images of the heart,
determining an internal myocardial power of the heart based on the plurality of images using the device, and
determining a remodeling of the heart when the internal myocardial power is higher than a predetermined threshold, in particular a threshold between 5 and 8 W/m², preferably a threshold between 6 and 7 W/m².

Preferably, the internal myocardial power is determined using a method of the first aspect or one of its implementations.

A fifth aspect of the present invention provides computer-readable storage medium storing program code, the program code comprising instructions that when executed by a processor carry out the method of the first aspect or one of its implementations.

Embodiments of the invention relate to the non-invasive diagnostic procedure characterizing individually myocardial internal and external power as well as respective myocardial power efficiency. In particular, embodiments relate to assessing the reduced myocardial efficiency due to congenital or acquired heart diseases such as coarctation of the aorta, bicuspid aortic valves, aortic or mitral valve stenosis and aortic or mitral valve regurgitation. These diseases cause additional load for the heart that could be initially compensated by the heart. With a disease development, heart remodeling is initiated and the myocardial efficiency is decreasing thus reducing the ability of the heart to provide additional power due to additional load caused by diseases as well as additional power required during stress conditions. An assessment of the myocardial efficiency, which requires an assessment of both internal and external myocardial power, allows assessing the individual response of the heart to the disease and thus could support time and type (interventional and/or pharmacological) of a treatment decision of a physician.

### BRIEF DESCRIPTION OF THE DRAWINGS

To illustrate the technical features of embodiments of the present invention more clearly, the accompanying drawings provided for describing the embodiments are introduced briefly in the following. The accompanying drawings in the following description are merely some embodiments of the present invention, modifications on these embodiments are possible without departing from the scope of the present invention as defined in the claims.

FIG. 1 is a block diagram illustrating a method in accordance with an embodiment of the present invention,

FIG. 2 is a block chart illustrating a number of optional features of embodiments of the present invention,

FIG. 3 is a schematic illustration of parameters used to estimate contraction time in accordance with the present invention,

FIG. 4 is a schematic illustration of a method to estimate inflow valve closing time in accordance with the present invention,

FIG. 5 is a diagram illustrating internal myocardial power values that have been determined with a method according to the present invention,

FIG. 6 is a diagram illustrating external myocardial efficiency values and circulatory efficiency values that have been determined with a method according to the present invention, and

FIG. 7 is a diagram illustrating a correlation of the proposed heart power analysis vs. established clinical standards for pressure-volume load assessment, thus showing an added value against an analysis of single clinical parameters.

### DETAILED DESCRIPTION OF EMBODIMENTS

The following description illustrates preferred embodiments of the present invention, the scope of the present invention is not limited to this. Any variations or replacements can be easily made through person skilled in the art. Therefore, the protection scope of the present invention should be subject to the protection scope of the attached claims.

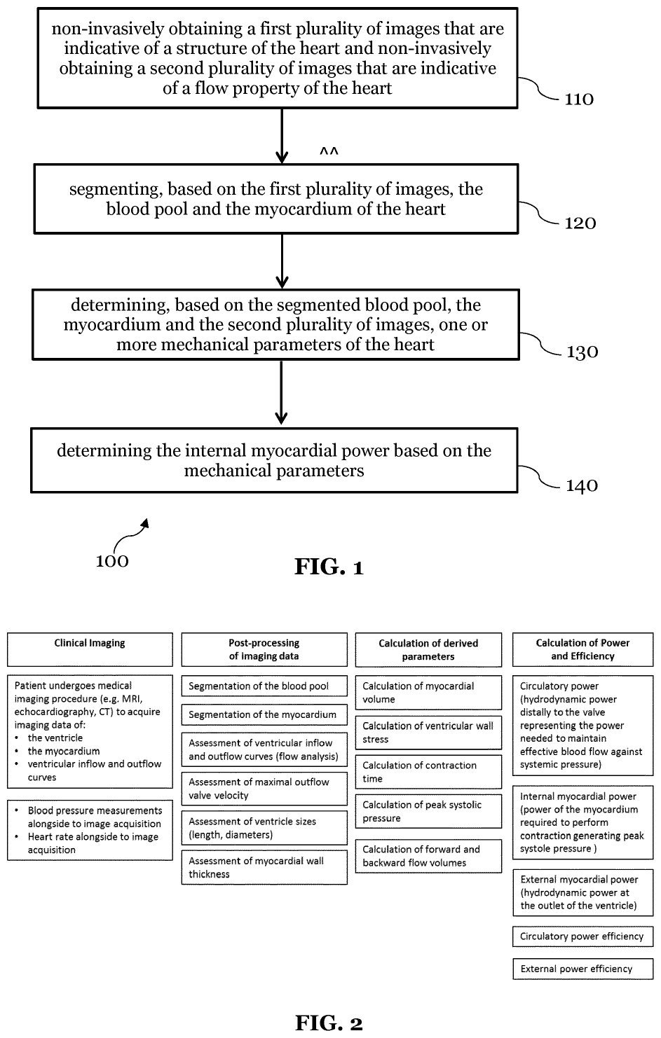

FIG. 1 is a block diagram illustrating a method in accordance with an embodiment of the present invention.

The method comprises a first step of non-invasively obtaining one or more first images that are indicative of a structure of the heart and non-invasively obtaining one or more second images that are indicative of a flow property of the heart. For example, the obtained images may be images that have been previously acquired using non-invasive images and stored on a workstation.

In a second step of the method, based on the one or more first images, the blood pool and the myocardium of the heart are segmented. It is understood that the segmentation of the blood pool and the myocardium may be also be based on further information, e.g. from the one or more second images.

In a third step, based on the segmented blood pool, the myocardium and the second images, one or more mechanical parameters of the heart are determined.

In a fourth step, the internal myocardial power is determined based on the one or more mechanical parameters.

FIG. 2 shows a block chart for diagnostic measures including four steps: clinical imaging accompanied with an assessment of heart rate and systemic blood pressures, post-processing of imaging data for anatomy and flow, calculation of derived parameters based on post-processing and finally calculation of the myocardial power and efficiency for clinical measures. The steps shall be explained in more detail in the following.

Clinical Imaging

Patients undergo imaging procedure allowing to acquire imaging data of the ventricle blood pool, myocardium as well as ventricular inflow and outflow conditions. MRI, CT and echocardiography or their combination are, among others, possible imaging modalities. 3D data are preferable. However, analysis could also be done based on 2D imaging data. Flow conditions can be assessed directly by methods like 4D MRI or Doppler echocardiography. Alternatively, they could be assessed indirectly by volumetric measurements of the ventricle (e.g. from 3D echocardiography or CINE MRT). Imaging acquisition is preferably accompanied by an assessment of the heart rate (HR) as well as the measurement of systemic blood pressure: 1) preferably using non-invasive methods (e.g. cuff-based measurements) or 2) can be performed based on invasive catheter-based measurements.

Post-Processing of Imaging Data

Post-processing can be performed by various interchangeable software tools. Several products are regularly available for routinely-used imaging devices or independent solutions can be used. The segmentation of the blood pool and the myocardium needs to be performed. Based on segmentations geometrical parameters of the ventricle and myocardium necessary to calculate the ventricular wall stress are assessed: ventricular radii, lengths and volumes, myocardial volume, myocardial wall thickness.

Preferably, epicardial and endocardial segmentations as well as LV volumetry and anatomical measurements are performed based on gapless balanced Turbo Field Echo (bTFE) cine two-dimensional short axis sequences 25. All images were analyzed using View Forum (Philips Medical Systems Nederland B.V; View Forum R6.3V1L7 SP1). The entire ventricle and the myocardium without the papillary muscles were segmented during diastole and systole. Nevertheless, other approaches including those based on other imaging modalities (e.g. echocardiography) and those for automatic segmentation (Xiu-Xia Luo et al. Echo Res Pract. 2017 December; 4(4): 53-61.) can be applied if clinical accuracy of measurements is achieved. New approaches with 3D echocardiography (Nikitin et al. Eur J Echocardiography (2006) 7, 365e372) were demonstrated to provided similar diagnostic accuracy as cardiac MRI and can be a possible alternative.

Flow analysis needs to be performed: inflow and outflow flow rate curves should be generated in order to measure contraction time. Flow curves could be measured directly based on flow imaging or indirectly based on dynamic assessment of geometries. Maximal outflow valve velocity should be measured to allow estimation of the valve pressure drop. Flow analysis was done using View Forum software (Philips Medical Systems Nederland B.V; View Forum R6.3V1L7 SP1), a software provided together with a MRI scanner used for imaging. Other software packages for flow analysis of functional 4D flow MRI sequences could be used equally.

Calculation of Derived Parameters

Derived parameters based on post-processed data are calculated: Myocardial volume is derived from myocardial wall segmentation or based on ventricular length together with myocardial wall thickness. If peak systolic ventricular pressure P_{sys} is not measured directly, P_{sys} should be derived indirectly. P_{sys} is the calculated sum of the systolic pressure in the aorta and the pressure gradient across the outflow valve, whereas the systolic pressure in the aorta is derived from the peripherally (e.g. arm) measured systolic pressure by taking into account the increasing from the heart pulse pressure. The pulse pressure increase could be calculated using in the literature published equations. The valve pressure drop is calculated by using the Bernoulli's equation. Ventricular wall stress σ_{wall} is calculated based on measured ventricular peak systolic pressure and geometric parameters of the myocardial wall. The simplest way is the calculation according to the Laplace law:

$${σ}_{wall} = {P}_{s y s} * \frac{{R}_{B P}}{2 * {S}_{wall}}$$

where S_{wall} is a mean myocardial wall thickness, P_{sys} is the peak systolic pressure in the ventricle and R_{BP} is the mean radius of the ventricular blood pool. The wall stress could also be calculated using other possible equations (e.g. thick wall Laplace law). These other equations may be based on the same kind of parameters, including S_{wall}, P_{sys}, and R_{BP}. Contraction time (t_{cs}) is the period between the start of ventricular contraction and peak systolic pressure and/or flow. If contraction time is not measured directly from ventricular pressure curves, t_{cs} should be derived indirectly: The sum of inflow valve closing time t_{CT}, isovolumetric contraction time t_{IVC} and ejection time t_{ET} define t_{cs}. The ejection time t_{ET} is derived from outflow curve. The isovolumetric contraction time t_{IVC} is derived from combined inflow and outflow curves. The sum of the inflow valve closing time t_{CT} and isovolumetric contraction time t_{IVC} is derived from ejection time t_{ET} together with systemic pressure values assuming ventricular pressure increase according to parabolic equation. In order to calculate internal myocardial power, the partial use of individual components (t_{cs}, t_{IVC}, t_{ET}) may provide further simplification with adequate diagnostic accuracy. Forward and backward flow volumes are derived from outflow curves or based on dynamic assessment of ventricular geometry.

Non-Invasive Assessment of Contraction Time

In the following, a preferred way of determining the contraction time t_{cs} using a non-invasive approach shall be explained in detail. Therein, the contraction time t_{cs} is defined as the period between the start of ventricular (myocardial) contraction at the end-diastolic pressure and a peak systolic pressure which is correlating with a peak aortic flow.

A known direct way of measuring the contraction time is based on catheter measured pressure curves that represent an invasive measurement associated with X-Ray radiation as well as a use of contrast agent. However, these are both associated with a risk for patients. As an invasive procedure, the heart power assessment using this approach is not available for a broad population of patients.

Instead, an indirect non-invasive approach allows determining the contraction time t_{cs} as the sum of three sequent periods: inflow valve closing time t_{CT}, isovolumetric contraction time t_{IVC} and ejection time t_{ET}:

t_{CS}=t_{CT}+t_{IVC}+t_{ET} (1)

The inflow valve closing time t_{CT} is a period of the increasing ventricle pressure necessary to stop the blood inflowing from the atrium into the ventricle, which is owning a momentum. The inflow valve closing time t_{CT} can be determined as a time at which two curves, a hyperbola and a parabola, are crossing. Therein, a hyperbola is a pressure curve p_{H} as a function of time t, [ms], e.g. as described by:

p_{H}=(25*1.05*(SV)^{2/3}*10⁵)/(133*T_{dia}*t), [mmHg] (2)

whereas SV is a stroke volume [ml] and T_{dia} is a diastole period [ms] calculated from heart rate HR [bpm] using e.g. the following equation:

T_{dia}=(60*10³/HR)−805*(0.18365317+0.54677315*e^{(-0.015150054*HR)}) (3)

The used parabola curve can be a pressure curve pₚ as a function of time t, [ms] as described by:

pₚ=P_{SYS}−C*(t−T₁)², [mmHg] (4)

whereas time T₁, [ms] is calculated e.g. according to following equation (5) and a parabola constant C e.g. according to following equation (6):

T₁=t_{ET}+t_{ET}*(P_{SYS}/(P_{SYS}−P_{DIA}))^{1/2} (5)

C=(P_{SYS}−P_{DIA})/(t_{ET})² (6)

P_{SYS} and P_{DIA}, [mmHg] are systolic and diastolic pressures. The ejection time t_{ET} is directly measured from outflow curve. The parabola equation is fully defined by three parameters: two pressure values P_{SYS} and P_{DIA} of the parabola based on cuff based measurements and a period t_{ET} between these two parabola points as measured from flow curve as well as an assigning of the point P_{SYS} to the parabola vertex.

The isovolumetric contraction time t_{IVC} can be directly measured from combined inflow and outflow curves.

Alternatively, the sum of the inflow valve closing time t_{CT} and isovolumetric contraction time t_{IVC} could be estimated from ejection time t_{ET} together with systemic pressure values P_{SYS} and P_{DIA}, assuming ventricular pressure increase according to parabolic equation (4).

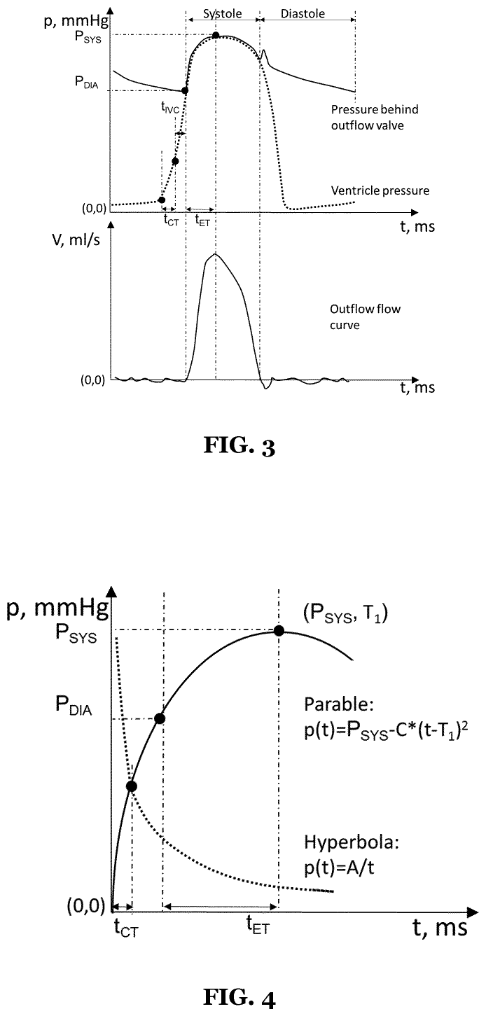

FIG. 3 provides a further illustration of parameters used to estimate the contraction time and FIG. 4 illustrates a method for estimating the inflow valve closing time. As illustrated in FIG. 4, the inflow valve closing time may be defined as a time when a first and a second pressure curve intersect, wherein the first pressure curve is modeled as a hyperbola p(t)=A/t, wherein A is a predetermined value, e.g. determined based on (SV)^{2/3}, and the second curve is modeled as a parabola p(t)=P_{SYS}−C*(t−T₁)², wherein P_{SYS} is the peak systolic pressure and C and T₁ are constant values, e.g. determined according to equations (5) and (6).

Calculation of Power and Efficiency

Internal myocardial power is defined as the power of the heart to perform myocardial contraction. Internal myocardial power IMP is preferably calculated using the following equation:

$$IMP = \frac{{V}_{wall} * {σ}_{wall}}{{t}_{CS}}$$

wherein V_{wall} is the myocardial wall volume, σ_{wall} the wall stress, and t_{cs} the contraction time of the ventricle. Internal myocardial power is indexed to BSA allowing inter-individual comparison.

Circulatory Power (CP) is defined as the hydrodynamic power after the ventricle outflow valve. The power needed to pump a given amount of blood against a given resistance characterized by systemic pressure. CP is calculated with the following equation:

CP=MAP*Q[W]

Therein, the mean arterial pressure (MAP) is assessed from measured peripheral systolic and diastolic pressure. Q is the effective Cardiac Output (CO_{eff}) and can be assessed from heart volumetry: CO_{eff}=(forward flow volume−backward flow volume)*HR (heart rate) or from outflow curves, depending on the imaging modalities used. The ratio between CP and IMP

$${Eff}_{C P} = \frac{C P}{I M P} * 100 [ \% ]$$

is defined as the Circulatory Efficiency (Eff_{CP}) of the heart. Eff_{CP} is the level of efficiency of the myocardium to provide effective cardiac output in the arterial system taking into account power loss in the myocardium and due to diseased heart valves.

External Myocardial Power is defined as hydrodynamic power at the outlet of the ventricle. It is the power needed to pump a given amount of blood against a given resistance and across a given gradient by a stenotic valve. EMP is computed by the following equation: EMP=(MAP+AoG)*COₜₒₜₐₗ. AoG is the aortic pressure gradient and COₜₒₜₐₗ (forward flow volume) multiplied by HR. The external myocardial efficiency is defined as ratio between EMP and IMP: is defined as the External Myocardial efficiency (Eff_{EMP}) of the heart:

$${Eff}_{EMP} = \frac{E M P}{IMP} * 1 \; 00 [ \% ]$$

It is the level of efficiency of the myocardium to provide effective cardiac output to arterial system and, additionally, to overcome all “resistances” such as the pressure gradients across stenotic valve.

Exemplarily Use of the Heart Power and Efficiency Analysis

The disclosed method can be applied in patients with different heart disease. Myocardial power was successfully shown to be able to quantify cardiac remodeling processes. Circulatory efficiency and internal myocardial power measurements gradually reflect disease response in patients with aortic valve stenosis (AS) and combine aortic valve stenosis and insufficiency (AS/AI), even when external myocardial efficiency and EF are still compensated.

In patients with AS (n=59), AS/AR (n=21) and controls (n=14) internal myocardial power, external myocardial efficiency and circulatory efficiency were computed from MRI volumetric as well as blood flow measurements and blood pressures. Data were compared between groups; sub-analysis was performed for patients with normal and reduced ejection fraction (EF). Median internal myocardial power was increased in AS, 7.7 W/m2 (interquartile range [IQR] 6.0-10.2; p=0.002) and AS/AR, 10.8 W/m2 (8.9-13.4; p<0.001) when compared to controls, 5.4 W/m2 (4.2-6.5), and was lower in AS than AS/AR (p<0.001). Circulatory efficiency was decreased in AS, 8.6% (6.8-11.1; p=0.008) and AS/AR, 5.4% (4.1-6.2; p<0.001) when compared to controls, 11.8% (9.8-16.9), and was higher in AS than AS/AR (p<0.001).

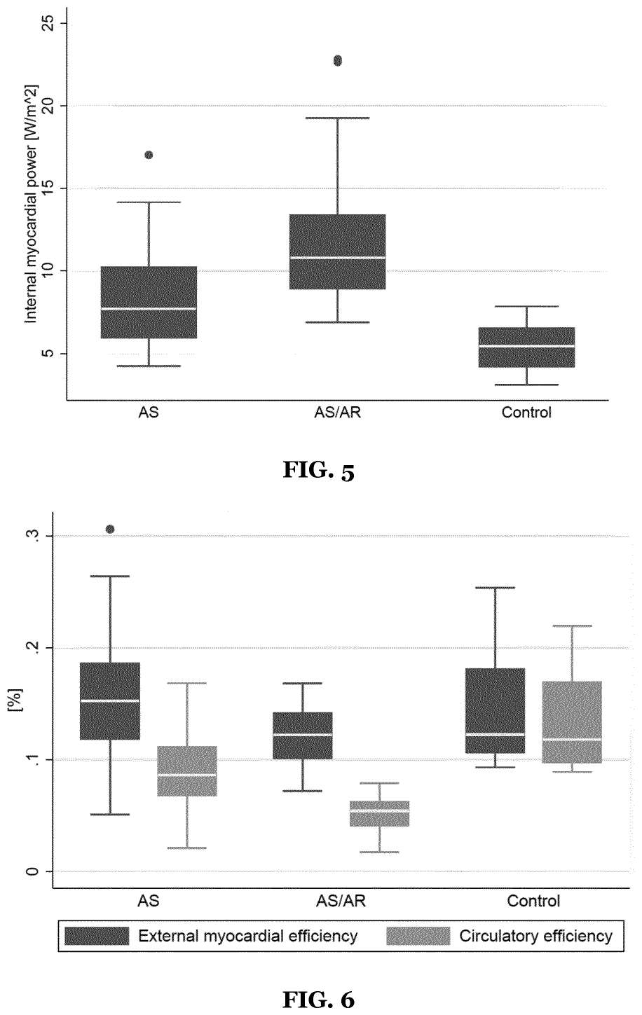

External myocardial efficiency was significantly higher in AS, 15.2% (11.9-18.6) than in AS/AR, 12.2% (10.1-14.2; p=0.011). External myocardial efficiency did not differ in patients with normal EF, 14.9% (12.9-17.9) when compared to controls 12.2% (10.7-18.1; p=0.218) and was significantly lower in patients with reduced EF, 9.8% (8.1-11.7; p=0.025). The results are illustrated in FIG. 5 and FIG. 6.

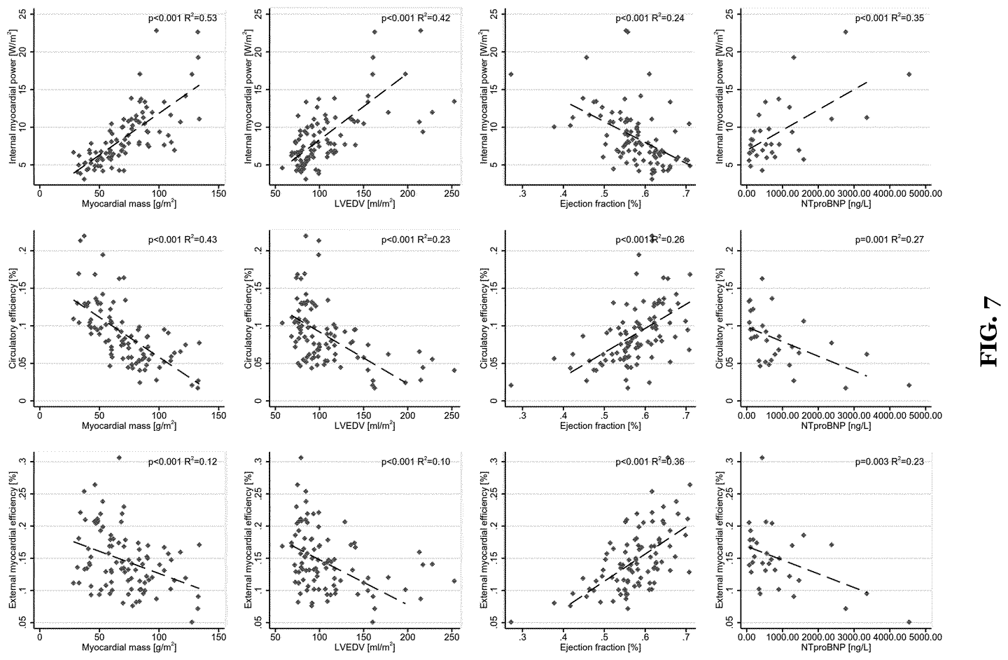

In AS and AS/AR, there was a significant positive correlation between N-terminal pro b-type natriuretic peptide (NT-proBNP) and internal myocardial power (R²=0.37, p<0.001). Furthermore, there were significant negative correlations between NT-proBNP and: (1) CircE (R²=0.29, p=0.001) and (2) EME (R²=0.25, p=0.003) in patients with AVD. Besides NT-proBNP, significant correlations between internal myocardial power, CircE and EME to established clinical standards for pressure-volume load assessment (myocardial mass, LVEDV and EF) can be found (shown in FIG. 7). While correlations were significant, the coefficient of determination (R²) was below 54% for internal myocardial power and below 36% for EME for all established parameters, with each of the single parameters only explaining some of the variability of the combined energetic measures.

In other words, looking at correlation to established clinical standards: In AS and AS/AR, there was a significant positive correlation between N-terminal pro b-type natriuretic peptide (NT-proBNP) and internal myocardial power (R²=0.37, p<0.001). Furthermore, there were significant negative correlations between NT-proBNP and: (1) CircE (R2=0.29, p=0.001) and (2) EME (R2=0.25, p=0.003) in patients with AVD. Besides NT-proBNP, significant correlations between internal myocardial power, CircE and EME to established clinical standards for pressure-volume load assessment (myocardial mass, LVEDV and EF) can be found (as shown in FIG. 7).

Hypertrophy was present in 32/80 (40%), dilatation in 37/80 (46%) and an EF below 50% in 12/80 (15%) patients. Receiver-operator characteristics (ROC) demonstrated internal myocardial power to detect differences between patients with at least one component associated to remodeling (regardless of the type: hypertrophy, dilatation or EF impairment) and those without. We found an area under the curve (AUC) of 90%. Using a cut-off of 6.8 W/m² we found the highest rate of correctly classified patients with a sensitivity of 96% and a specificity of 63% to detect remodeling. We found 14 patients without conventional remodeling above this cut-off, of which 90% had clinical symptoms during ordinary activity/less than ordinary activity or resting conditions, compared to 70% in the conventional remodeling group (p=0.128). Looking at hypertrophy, dilatation and ventricular function alone would have left 18% of symptomatic patients undetected.

If the IMP value is higher than 6.8 W/m², based on our current analyses remodeling was detected with a sensitivity of 96% and a specificity of 63%. Even if conventional age and gender-specific reference values for established cardiac remodeling parameters are not exceeded, IMP and the resulting circulatory efficiency can help to detect symptomatic patients with elevated heart power above a given cut off. Of all vascular heart disease patients analyzed to date 18% of symptomatic patients would have remained undetected by single parameter cut-offs only. Since current guidelines in cardiovascular disease usually consider the presence of symptoms and the associated risk for arrhythmia heart failure and sudden cardiac death a relevant information for clinical decision making processes.

1. A method for determining an internal myocardial power of a heart, the method comprising:
non-invasively obtaining one or more first images that are indicative of a structure of the heart,
non-invasively obtaining one or more second images that are indicative of a flow property of the heart,
segmenting, based on the one or more first images, the blood pool and the myocardium of the heart,
determining, based on the segmented blood pool, the myocardium and the one or more second images, one or more mechanical parameters of the heart, and
determining the internal myocardial power based on the one or more mechanical parameters.

2. The method of claim 1, further comprising, during a non-invasive acquisition of the first and second images, acquiring a heart rate and/or a systemic blood pressure.

3. The method of claim 1, wherein
the one or more first images are acquired using MRI, CT and/or echocardiography, and/or
the one or more second images are acquired using 4D flow MRI, 2D flow MRI and/or Doppler echocardiography.

4. The method of claim 1, wherein the one or more mechanical parameters include a contraction time of the heart, wherein the contraction time is determined based on a sum of an inflow valve closing time, an ejection time, and an isovolumetric contraction time.

5. The method of claim 4, wherein
the inflow valve closing time is determined as a time when a first and a second pressure curve intersect, wherein the first pressure curve is modeled as a hyperbola and the second curve is modeled as a parabola,
the ejection time is determined based on an outflow curve that is determined based on the one or more second images, and/or
the isovolumetric contraction time is determined based on a combined inflow and outflow curve.

6. The method of claim 1, wherein the one or more mechanical parameters include a ventricular radius, a length, a volume, a myocardial volume, and/or a myocardial wall thickness of the heart.

7. The method of claim 1, wherein the internal myocardial power that is determined based on a volume of the myocardial wall volume V_{wall}, a wall stress σ_{wall}, and a contraction time of the ventricle t_{cs}, based on the equation
$$IMP = \frac{{V}_{wall} * {σ}_{wall}}{{t}_{C S}} .$$

8. The method of claim 1, further comprising determining a circulatory power based on a mean arterial pressure and an effective cardiac output, based on a product of mean arterial pressure and effective cardiac output.

9. The method of claim 1, wherein the one or more mechanical parameters include a myocardial volume, a ventricular wall stress, a peak systolic pressure, a forward flow value and/or a backward flow volume.

10. The method of claim 1, wherein the method further comprises:
non-invasively obtaining a systemic blood pressure based on a non-invasive blood pressure measurement, based on a sphygmomanometer or oscillometric approach, and
determining, based on a flow property of the heart and the systemic blood pressure, one or more contraction time parameters of the heart,
wherein the determining the internal myocardial power is based on the one or more mechanical parameters, a myocardium volume of the heart and the one or more contraction time parameters of the heart.

11. A method for determining a remodeling of the heart of a patient, wherein the method comprises:
reading a plurality of images of the heart,
determining an internal myocardial power of the heart based on the plurality of images, and
determining a remodeling of the heart when the internal myocardial power is higher than a predetermined threshold.

12. The method of claim 11, wherein the internal myocardial power is determined using a method comprising:
non-invasively obtaining one or more first images that are indicative of a structure of the heart,
non-invasively obtaining one or more second images that are indicative of a flow property of the heart,
segmenting, based on the one or more first images, the blood pool and the myocardium of the heart,
determining, based on the segmented blood pool, the myocardium and the one or more second images, one or more mechanical parameters of the heart, and
determining the internal myocardial power based on the one or more mechanical parameters.

13. A device for determining an internal myocardial power of a heart based on non-invasive imaging, the device comprising:
an image obtaining unit configured to obtain one or more first images of the heart that are indicative of a structure of the heart and one or more second images that are indicative of a flow property of the heart,
a segmentation unit configured to segment, based on the one or more first images, the blood pool and the myocardium of the heart,
a parameter determining unit configured to determine, based on the segmented blood pool and the myocardium and the one or more second images, one or more mechanical parameters, and
a calculation unit configured to determine the internal myocardial power based on the mechanical parameters.

14. The device of claim 10, wherein the image obtaining unit comprises a network interface configured to obtain the first images and/or the second images from a remote device in the network, in particular from an MRI, CT or echocardiography machine.

15. Use of the device of claim 13 for determining a remodeling of the heart of a patient, comprising steps of
reading a plurality of images of the heart,
determining an internal myocardial power of the heart based on the plurality of images using the device, and
determining a remodeling of the heart when the internal myocardial power is higher than a predetermined threshold, in particular a threshold between 5 and 8 W/m², preferably a threshold between 6 and 7 W/m².

16. The use of claim 15, wherein the internal myocardial power is determined using a method comprising:
non-invasively obtaining one or more first images that are indicative of a structure of the heart,
non-invasively obtaining one or more second images that are indicative of a flow property of the heart,
segmenting, based on the one or more first images, the blood pool and the myocardium of the heart,
determining, based on the segmented blood pool, the myocardium and the one or more second images, one or more mechanical parameters of the heart, and
determining the internal myocardial power based on the one or more mechanical parameters.

17. A computer-readable storage medium storing program code, the program code comprising instructions that when executed by a processor carry out the method of claim 1.

18. A computer-readable storage medium storing program code, the program code comprising instructions that when executed by a processor carry out the method of claim 11.

19. The method of claim 11, wherein the predetermined threshold is between 6 and 7 W/m².

20. The device of claim 14, wherein the device further comprises a user interface for a visual verification of the segmentation by a physician.
